(19) United States
(12) Patent Application Publication
(10) Pub. No.: US 2013/0211424 A1
(43) Pub. Date: Aug. 15, 2013

(54) ADJUSTABLE FIXATION SYSTEM FOR NEUROSURGICAL DEVICES

(71) Applicants: Jean Philippe Thiran, Granges (Veveyse) (CH); Claudio Pollo, Lausanne (CH); Rémi Charrier, Crissier (CH); David Haag, Verrieres (CH)

(72) Inventors: Jean Philippe Thiran, Granges (Veveyse) (CH); Claudio Pollo, Lausanne (CH); Rémi Charrier, Crissier (CH); David Haag, Verrieres (CH)

(73) Assignees: Universite de Lausanne, Lausanne (CH); Ecole Polytechnique Federale de Lausanne, Lausanne (CH)

(21) Appl. No.: 13/811,827

(22) PCT Filed: Jul. 23, 2010
(86) PCT No.: PCT/IB10/53359

§ 371 Date: Apr. 25, 2013

(51) Int. Cl. A61B 19/00

(52) U.S. Cl. CPC A61B 19/203
USPC 606/130

(57) ABSTRACT

The system comprises at least a support (5) to be attached to a patient by fixing means (4), said support having an outer thread (7), a first nut (9) with an inner thread (11) cooperating with said outer thread (7); and an anchor having a spherical bottom end (10) being placed between said support and said nut, a spherical head (15) said spherical bottom end and head being linked by a shaft, a second nut (16) having an inner thread (17) and intended to be screwed on an external device having a corresponding thread (31), such as a platform (20) or a surgical device (30), whereby the tightening of at least one of the nuts (9,16) on the support and/or on the external device allows to block the position of the anchor.

### FIELD OF THE INVENTION

The present invention relates to neurosurgery and to devices used in this field. More specifically, the present invention concerns a removable and adjustable fixation system for neurosurgical devices.

### STATE-OF-THE-ART

Stereotactic surgery, also called stereotaxy, is a minimally-invasive form of surgical intervention which makes use of a three-dimensional coordinates system to locate targets inside the body and to perform on them some action such as ablation (removal), biopsy, lesion (thermo-lesion, X-Ray or Gamma-ray induced lesion), injection, electrical stimulation, implantation, etc.

“Stereotactic” in Greek (another accepted spelling is “stereotaxic”) means movement in space.

In neurosurgery, stereotactic procedures refer to the use of a reference frame, a mechanical device equipped with head-holding clamps and bars which puts the head in a fixed position in reference to the coordinate system (the so-called zero or origin) of the frame. Each point in the brain can then be referenced by its three coordinates (x, y and z) in an orthogonal frame of reference (Cartesian coordinates), or, alternatively, a polar coordinates system, also with three coordinates: angle, depth and antero-posterior location. The standard way of defining target points in stereotactic neurosurgical procedures consists in imaging the patient's head in three dimensions (3D) by Computed Tomography (CT) or Magnetic Resonance Imaging (MRI) while holding the stereotactic frame (or a subset of it). Since both the brain and the frame are visible on the images, the coordinates of the target point can be defined in the coordinate system of the frame, either directly on images when the target is clearly identifiable or with the help of stereotactic atlases. Finally, guide bars in the x, y and z directions (or alternatively, in the polar coordinate holder), fitted with high precision scales, allow the neurosurgeon to reach the target with a probe (electrode, needle, cannula, X-ray or Gamma-ray beam, etc.) inside the brain, at the calculated coordinates for the desired structure, following an optimal trajectory through a small twist drill in the skull.

The main advantage of this procedure is its high precision.

Moreover, stereotaxy is a classical procedure in neurosurgical practice: every neurosurgeon is trained for this procedure.

Its main drawbacks are clear though:
the feeling of pressure and pain during and after the placement of the frame, which has to be held by the patient for several hours, all along the imaging and surgical procedures,
the immobilization of the head at the OR table during the surgical procedure, that may result in a discomfort due to the duration of some operations (several hours) and in a highly unsuitable displacement of the frame on the patient's head if he/she tried to move during the operation, leading to a severe loss of precision in the probe placement,
potential artifacts in MR imaging performed with stereotactic frame, due to the distortion of the magnetic field of the MR scanner, induced by the frame, that may lead to loss of precision in targeting on such images.

Therefore researchers have tried to propose alternatives to stereotactic frames, while keeping the high precision as a strict requirement.

The main efforts have been made in the combination of imaging and neurosurgical robots: small fiducial markers are placed on the skull of the patient. The 3D imaging (either CT or MRI) is done, and adequate calibration/registration procedure is then used to register the coordinate system of a robot arm with the coordinate system of the patient head, defined by the fiducial markers. Then the robot arm can be placed in a pre-defined position and orientation defined with respect to the patient head, and either serve as a guide/support for inserting the surgical tools (needle, electrode, etc) or doing the insertion itself (drilling, etc) under the control of the neurosurgeon. While it partially fulfills the precision requirements and minimizes the patient discomfort, the main drawbacks of such systems are the high level of complexity in using, calibrating and maintaining them, and most importantly their prohibitive cost, which makes such systems only affordable for a very limited number of hospitals in the world. Finally, there is an intrinsic procedural danger in having a robot moving independently of the head. For all those reasons, neurosurgical robot systems are not competitive as compared to classical stereotactic frames.

At the beginning of the years 2000, the Vanderbilt University Medical Centre, Nashville, Tenn., introduced the STarFix technology which consists in a patient-specific tripod, specially realized for each patient. The procedure is as follows: based on the intended entry area location, anchors, similar to fiducial markers, are screwed on the patient's head, and the patient is then scanned by CT or MRI. Then surgical planning software is used to define the target point with respect to the coordinate system defined by the anchors. Then the corresponding data are sent (by Internet) to the manufacturer (FHC Inc. Bowdoinham, Me., USA) which realizes a personalized tripod, called the STarFix. This tripod is then fixed on the patients head using the anchors. Guiding tools are fixed to the tripod to realize the operation. More details can be found on the internet at http://www.fh-co.com/p67-69B.pdf

The advantages of this procedure are its low complexity and increased comfort of the patient, as well as its compatibility with several guidance and tool holding devices. Other advantages include the precision (similar to the frame-based procedure), simplicity and efficiency.

But there are several important drawbacks:

A new tripod has to be realized by the manufacturer for each surgical operation. The realization takes between 1 and 3 days. Moreover the company is located in the USA, which may induce additional delays in shipping the tripod from the manufacturer to the user when the patient undergoes surgery in another country or continent.

Accordingly, during this period, the anchors have to stay implanted on the patient's head, which may cause pain and potential infections.

Most importantly, once the tripod is realized, there is no way to modify the surgical planning: the tripod being strictly based on the pre-operative planning, the trajectory can absolutely not be changed during the operation, to adapt to unexpected events.

Any change in the planning would require the realization of a new tripod, i.e. another 1 to 3 day delay with the drawbacks mentioned above if the patient is not in the USA.

Typical examples of stereotaxy devices are described in the following publications: WO 2004/058086, US 2006/0192319, U.S. Pat. No. 6,282,437, WO 2005/039386, WO 95/13758, WO 2007/095917, US 2007/106305 and WO 2007/031314.

In a recent development subject of PCT application No. WO 2009/060394 (the content of which is incorporated by reference in its entirety in the present application), the present Applicant has created a new adjustable stereotactic device and method for frameless neurosurgical stereotaxy. The system comprises at least three anchors intended to be attached to the patient and equipped with markers, an insertion guide device with an insertion guide intended to be attached to said anchors, an external calibration device with at least three calibration markers corresponding to said markers and a planning and imaging software. The external calibration device is patient-independent so that it may be reused for different patients. The planning and imaging software is used to determine the position of a target point in the patient with respect to the markers, the calibration device is used to calibrate and orient the insertion guide of the insertion guide device mounted on said calibration markers using the determination of the software before the insertion guide device is mounted on the anchors attached to the patient.

As one will understand, the fixation of the device on the head of the patient is important. On the one hand, the anchors are used to attach the insertion guide to the patient but also they are used as markers for building a referential in the imaging system.

An aim of the present invention is therefore to provide a fixation system that is easy to use and that can provide the necessary markers in the imaging system.

Per se, the use of anchors and markers is known in the art, for example from WO 2009/060394. As another example, US 2003/012043 (incorporated by reference in the present application) discloses a positioning fixture used to position a portion or all of a patient's body relative to a medical apparatus. According to the method described a set of bone anchors is attached to the patient's skull prior to scanning the patient. The anchors become the mounting locations on the patient's body which provides the means by which the patient's head will later be held in position in the radiotherapy apparatus. Each bone anchor has a threaded opening for accepting threaded bolts or other threaded attachments and prior to scanning each threaded opening is used to accept a scanning marker which comprises a threaded section attached to a marker portion. Said marker portion includes a material that will result in a visible image in the scanned image.

Other prior art publications include the following documents (by application field):

Guiding devices: U.S. Pat. No. 4,931,056, WO 2009/149398, WO 2008/153975, WO 2005/046451, WO 01/78814, US 2004/0243146, WO 99/16374, WO 2008/14261.

Markers and anchors: US 2004/0030237, US 2004/0030236, U.S. Pat. No. 5,013,316, WO 00/01316, US 2003/0125743, US 2004/0167391, WO 2004/075768, WO 2004/089231.

Surgery apparatus: EP 0 326 768, EP 0 207 452, US 2002/0052610.

General anchoring devices: EP 1 839 606, WO 96/08206, US 2006/0217713, EP 0 611 557, U.S. Pat. No. 5,269,784, US 2003/0229349, US 2005/0277925, WO 00/40167.

### GENERAL DESCRIPTION OF THE INVENTION

The present invention introduces a new way and system for rigidly fixing a neurosurgical device on a patient, for example on the head of a patient.

The invention is based on a minimum of three fixation systems which are attached to the skull of the patient using medical screws or other equivalent means. Each fixation system comprises at least a support and an anchor. The support is the part fixed in the skull with a medical screw. The anchor is a mobile part which goes on the support. The anchor is composed of an inferior part, a superior part and preferably two nuts, one being used to block the anchor in a desired position.

Each anchor is able to move in the space by describing a spherical movement. Due to the fact that each anchor has such an adaptation capability, it allows placing the fixation systems on the head without an extreme precision. The numerous degrees of freedom of the anchors allow attaching a neurosurgical device (or another device) by orienting each of these in order to match the precise geometry of its platform.

Before locking the complete system using anchors nuts, the neurosurgical device can be moved in order to perform a fine adjusting of its placement. The device has approximately three degrees of freedom: two in translation and one in rotation.

Once locked, the device is rigidly attached to the skull.

The invention in addition has the capability to allow removing the superior part of the anchor without losing the precise location of the device. This performance minimizes the risks linked to shocks on the system and increases the patient comfort during a waiting phase.

Another practical consequence of this capability is the fact that before the neurosurgical intervention, new sterilized anchor superior parts can be mounted through a sterile field on the remaining parts on the skull in order to provide a perfectly clean interface for the neurosurgical device.

The invention and its different embodiments will be better understood by the following description of illustrative examples.

### BRIEF DESCRIPTION OF THE DRAWINGS

FIG. 1 illustrates the use of a gauge used to place approximatively the supports on the head of the patient;

FIGS. 2A and 2B illustrate the different components of one fixation system in perspective view (FIG. 2A) and in cut view (FIG. 2B);

FIG. 3 illustrates the platform used to position the anchors at a correct position from each other;

FIG. 4 illustrates the possible movements of one fixation system when it is not tightened;

FIG. 5 illustrates the possible movements of the platform when the fixation systems are not tightened;

FIG. 6 illustrates the fixation systems with the superior parts removed;

FIG. 7 illustrates the fixation systems with the superior parts mounted;

FIG. 8 illustrates a surgical device mounted on the fixation systems;

FIG. 9 illustrates the surgical device seen in perspective;

FIG. 10 illustrates alternative parts of the system.

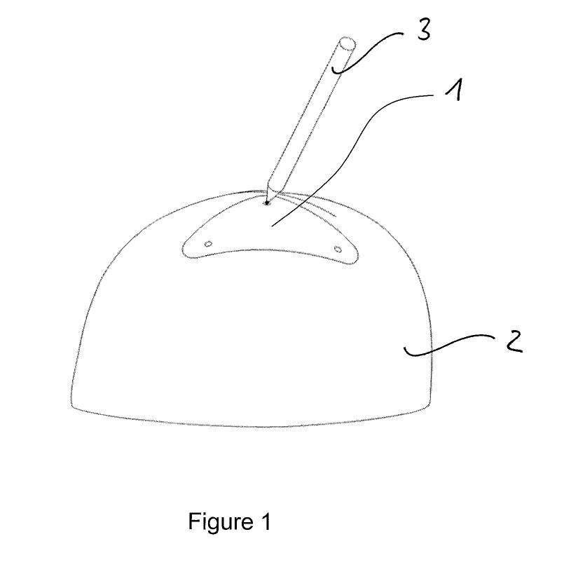

### DETAILED DESCRIPTION OF THE INVENTION

The device may comprise at least a gauge 1 which is used to mark the head 2 (skull) of the patient at least approximatively where the supports will be attached to the patient. This is schematically illustrated by pencil 3.

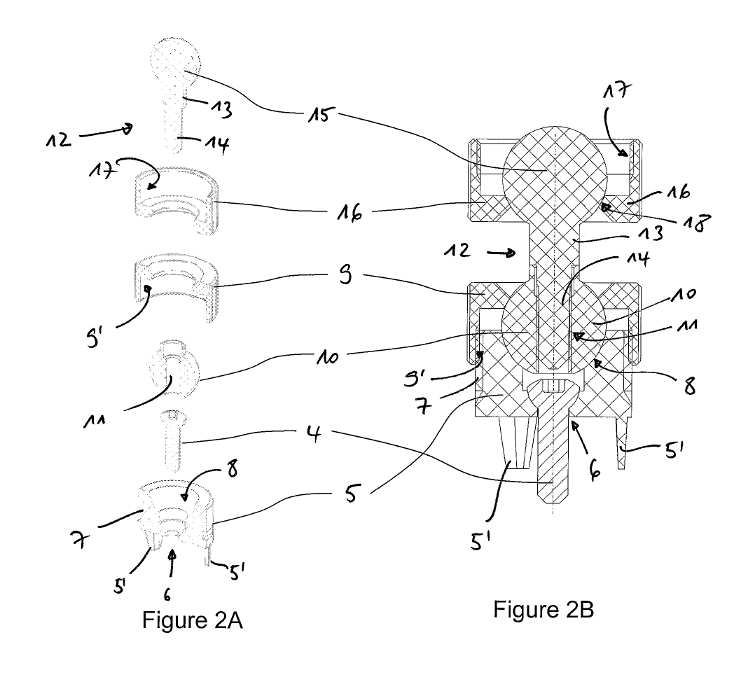

FIGS. 2A and 2B illustrates in a more detailed manner the elements and fixation systems used as anchors for the platform that will be attached to them (on order, for example, to carry out a surgical procedure).

Each fixation system comprises at least a screw 4 intended to be screwed in the patient (for example in his head) and to fix a base support 5 on the patient through an opening 6 of the support. The support 5, for example, has a cylindrical shape and comprises on its upper end (away from the patient) an outer thread 7 and several (for example three) pins 5′, said pins 5′ penetrating the skin and making contact with the bone (i.e. the skull) of the patient. The length of said pins 5′ is greater than the thickness of the skin in order to avoid compressing the skin. The support 5 also comprises a spherical cavity 8 (or at least a cylindrical cone shaped cavity) the use of which will be explained hereunder and a support nut 9 having an inner thread 9′ which cooperates with the outer thread 7 of the support 5. Of course, the support may have another shape but still an outer thread the use of which will be explained hereunder.

The anchor comprises at least a bottom part 10 which has a spherical shape with an inner thread 11, said part 10 being held between the support 5 (in its cavity 8) and the nut 9 when said nut is mounted on the support 5 by screwing. When in this position, the bottom part is able to rotate until its movements are blocked by a further tightening of the nut 9 on the thread 7 of the support 5.

When the lower nut 9 is not tightened, it is thus possible to orient the anchor by rotation and the anchor has three degrees of freedom in rotation, but only two are used (the spin is not relevant in the present configuration). The anchor may then be blocked in a chosen position. This is illustrated in schematical way in FIG. 4 with the arrows illustrating the degrees of freedom of the anchor, the rotation allowing an orientation in all directions (not only the two illustrated by the arrows).

The anchor in addition (see FIGS. 2A and 2B) comprises a top part 12 having a shaft 13 with a thread 14 and a spherical head 15. The thread 14 is intended to cooperate with the thread 11 of the bottom part by screwing one part into the other one thus forming the anchor with two spherical ends linked by the shaft 12.

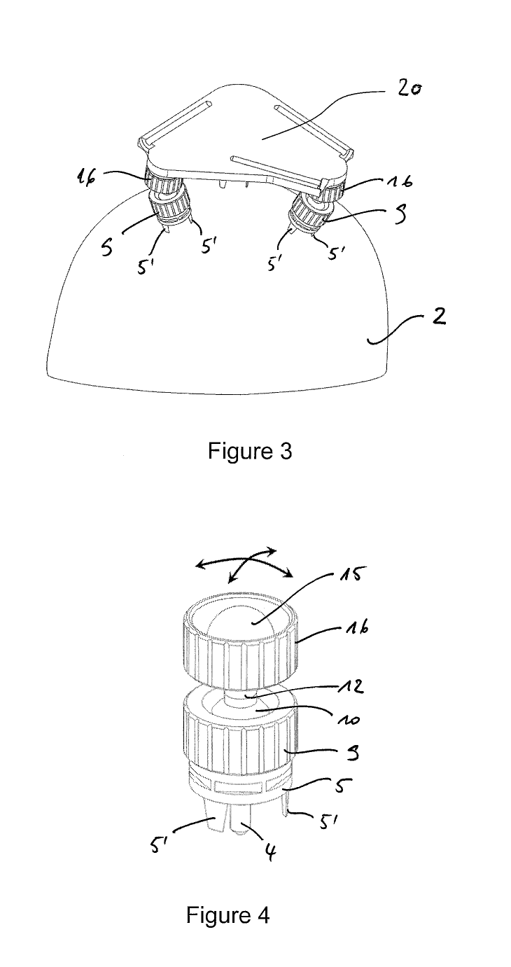

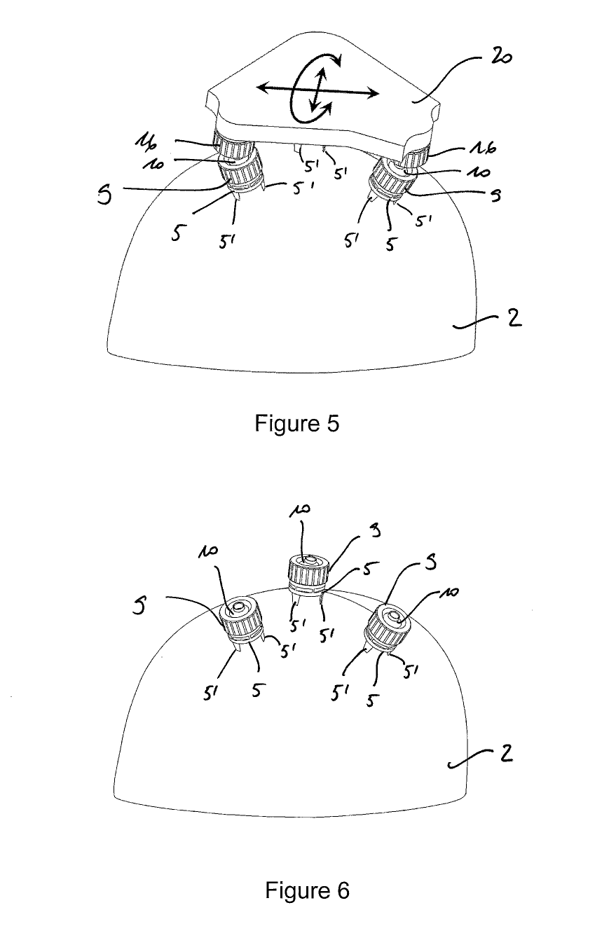

The fixation system further comprises a base nut 16 with an inner thread 17, said nut 16, in a manner similar to the nut 9, being used to attach a platform 20 to the anchors (as illustrated in FIGS. 3 and 5 for example). More specifically, the platform 20 used in combination with the anchors of the present invention comprises supports similar to support 5 described above, with an outer thread intended to cooperate with the inner thread 17 of the base nut 16, said support of the platform 20 comprising also a spherical cavity 18 such that it is possible to fix the platform on the anchors by screwing said nut 16.

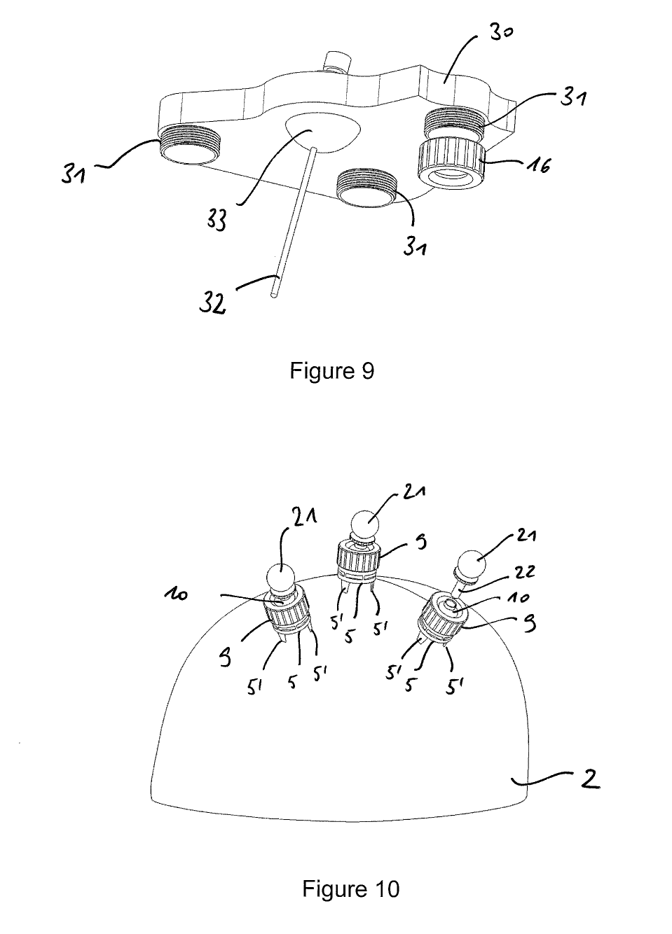

When the three or more fixation systems with anchors as described previously have been fixed on the head of the patient, the platform 20 compatible with the neurosurgical device platform 30 (see FIGS. 8 and 9) is mounted and screwed on the superior part of the anchor (see FIGS. 3 and 5). This allows positioning the anchors in the correct relative position from each other. At this point, when the system is not tightened, the platform 20 can be moved according three degrees of freedom: two in translation and one in rotation (see FIG. 5) (the movements induces small displacements in other directions but these are not relevant).

Once the nuts 9 and 16 are tightened, the position of the platform and anchors is kept.

The platform preferably comprises markers that can be seen on pre-operative image(s) taken of the patient carrying the fixation systems and the platform 20, said markers defining a reference system. This thus allows the user to define in the pre-operative image(s) the position of the target point in the patient in the reference system formed by the markers of the platform 20 and then reproduce precisely this target point in the calibration device (according to the principles disclosed in WO 2009/060394.

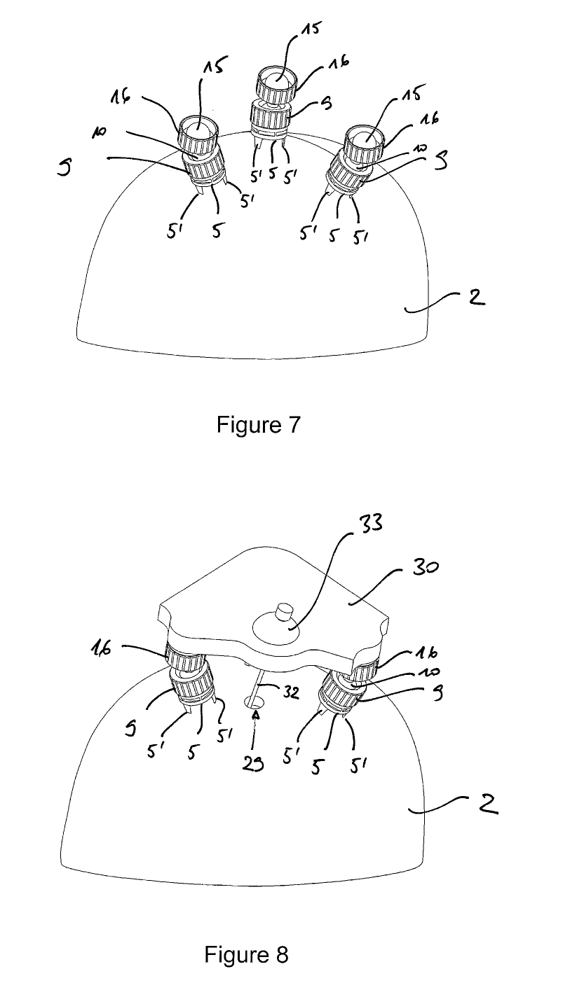

As mentioned above, once the locking has taken place (at least of the nuts 9), it is possible to remove the platform 20 without losing the precise location of the device, more specifically of the anchors. Only the nuts 16 are untightened which action frees the platform 20 whereas the lower nuts 9 are kept tightened so that the orientation of the anchors is maintained. This configuration is illustrated in FIG. 7.

The platform 20 or another device (with a corresponding platform, for example a surgical device) may then be replaced on the patient in a proper position at a later stage.

FIG. 6 illustrates a configuration with three supports 5 have been attached to the skull of a patient and the top parts of the anchors have been removed by unscrewing the spherical heads 15. If the nuts 9 have been tightened, the bottom parts remain oriented. In such a configuration, it is possible for example to attach other elements to the supports 5, for example markers 21 as illustrated in FIG. 10 and explained in more detail hereunder.

Alternatively, in order to be provide a sterile interface, the superior parts (top part 12 with shaft 13 and thread 14 and spherical head 15) of the anchors and the nut 16 can be removed and replaced by sterile ones (see FIGS. 6 and 7). This is typically the case when a surgical intervention is to be carried out.

To perform the intervention, a neurosurgical device 30 can simply be attached to the blocked anchors (see FIGS. 8 and 9) as described above. More specifically, as described, the platform 20 is used for example to define a position of the anchors (see the description in relation to FIG. 5, then the anchors are blocked by tightening of nuts 9. The surgical intervention may then be carried out as planned with the calibration device disclosed in WO 2009/060394.

The platform may then be removed and replaced by markers 21, for example metallic markers or plastic markers covered with a metallic layer (or at least markers that are visible on images produced by the imaging system used according to application WO 2009/060394 cited above and incorporated in the present application) that are identical to the top parts 12 discussed above. The markers may also comprise other passive or active features (such as diodes) in order to be detectable by a camera for example. Since the nuts 9 are tightened, the position of the markers is identical to the position of the parts 12. Typically this is done by unscrewing the threads 11 and 14 (see FIGS. 2 and 10) and screwing thread 22 of the marker 21 into thread 11. It is then possible to make pictures of the patient with the markers according to the principle described in WO 2009/060394 with the configuration illustrated in FIG. 10.

As mentioned previously, FIGS. 8 and 9 illustrate an example of a surgical device that can be used with the anchors of the present invention. The device may comprise a platform 30 with several threads 31 which cooperate with the nuts 16 of the anchors. The platform 30 may also carry a tool 32 through an insertion guide 33 which is used to orient the tool 32 toward the target point (not illustrated) through an opening 29 in the skull 2.

Also, the supports according to the present invention may be used in the stereotactic device described in WO 2009/060394, this application being incorporated in its entirety in the present application in this respect. As described in said publication, the disclosed system comprises the use of a patient independent calibration device which allows the calibration of the surgical apparatus before its effective use.

The invention is not limited to the systems and methods discussed above and modifications are possible, for example by using equivalent means.

Preferably, the support 5 and the screw 4 are made of biocompatible materials: these parts are the only ones in direct contact with the patient. All the other parts are made of suitable materials in the medical field or other field of use of the present object.

In addition, the nuts 9, 16 and corresponding outer threads 7, 31 may be replaced by equivalent means, such as for example a bayonet connector system.

Also, the illustrative examples given concern a neurosurgical intervention, but of course other surgical and non-surgical applications are possible with the disclosed devices and methods of the invention and this description should not be construed as limiting in this respect.

Finally, the present invention can be used as a kit comprising several elements together from which the user can choose. Such kit may contain one or several fixation system mounted or in spare parts, a platform, a calibration device, one or several markers. Any variant is of course possible in the frame of the present invention.

### REFERENCE NUMBERS

1 gauge

2 skull of a patient anchor

3 pencil

4 screw (for the anchors)

5 support

5′ pin

6 opening

7 outer thread

8 cavity

9 nut

10 spherical bottom part

11 inner thread

12 top part

13 shaft

14 inner thread

15 spherical head

16 nut

17 inner thread

18 cavity

19 -

20 platform

21 marker

22 thread

29 opening (of the skull)

30 surgical platform

31 thread

32 surgical tool

33 insertion guide

1. A fixation system, for example for a stereotactic device, said system comprising at least
a support to be attached to a patient by fixing means, said support having an outer thread,
a first nut with an inner thread cooperating with said outer thread;
and an anchor having a spherical bottom end being placed between said support and said nut, a spherical head said spherical bottom end and head being linked by a shaft,
whereby the tightening of the first nut on the support allows to block the position of the anchor.

2. A fixation system as defined in claim 1, wherein the anchor is made of a first part being formed by said bottom end in which a inner thread is formed, a second part being formed by said spherical head having a shaft the end of which comprise an outer thread, said two parts being assembled by the screwing of said thread in said inner thread.

3. A fixation system as defined in claim 1, wherein said support comprises a cavity for receiving said spherical bottom end.

4. A fixation system as defined in claim 1, wherein the support comprises at least three pins.

5. A fixation system as defined in claim 1, wherein said anchor is detectable by a camera.

6. A fixation system as defined in claim 1, wherein it comprises a second nut having an inner thread and intended to be screwed on an external device having a corresponding thread, such as a platform or a surgical device, whereby the tightening of the second nut on the external device allows to block the position of the anchor head.

7. A kit comprising at least one fixation system as defined in claim 1.

8. A kit as defined in claim 7 comprising a platform.

9. A kit as defined in claim 8, wherein said platform comprises markers defining a reference system.

10. A kit as defined in claim 7, wherein it further comprises a calibration device.

11. A stereotactic device comprising at least a kit as defined in claim 7 and a surgical device.
